(19) United States
(12) Patent Application Publication
(10) Pub. No.: US 2021/0393507 A1
(43) Pub. Date: Dec. 23, 2021

(54) EXTEMPORANEOUS COSMETIC AND/OR DERMATOLOGICAL PREPARATIONS

(71) Applicant: UNIVERSKIN, Nice (FR)

(72) Inventors: Charalampos ARAMPATZIS, Nice (FR); Claudine MASSON, Nice (FR)

(73) Assignee: UNIVERSKIN, Nice (FR)

(21) Appl. No.: 17/349,315

(22) Filed: Jun. 16, 2021

Related U.S. Application Data
(62) Division of application No. 15/311,066, filed on Dec. 22, 2016, now abandoned.

(30) Foreign Application Priority Data
May 13, 2014 (FR) 14.01067

(51) Int. Cl. A61K 8/92; A61K 8/34; A61K 8/64; A61Q 19/02; A61Q 19/08; A61K 8/27; A61K 8/36; A61K 8/49; A61K 8/60; A61K 8/66; A61K 8/67; A61K 8/73; A61Q 19/00

(52) U.S. Cl. CPC A61K 8/922; A61K 2800/28; A61K 8/64; A61Q 19/02; A61Q 19/08; A61K 8/34; A61K 8/27; A61K 8/361; A61K 8/498; A61K 8/602; A61K 8/66; A61K 8/673; A61K 8/675; A61K 8/676; A61K 8/73; A61Q 19/00; A61K 8/345

(57) ABSTRACT

The present invention relates to an extemporaneous product for the preparation of dermatological and/or cosmetic compositions, this product comprising, on the one hand, one or several active substances and on the other, a universal base allowing such active substance or substances to be solubilised and comprising at least one oil rich in omega-3, water, glycerine, an alcohol other than glycerine, and emulsifier and a biomimetic peptide.

The technical scope of the present invention is that of cosmetics and dermatology and more particularly that of cosmetic and/or dermatological compositions prepared extemporaneously. These compositions may namely contribute to the general aesthetic improvement of the skin of the body or face.

Dermatologists, cosmetic doctors and chemists are confronted with very diverse cutaneous pathologies and problems. These include acne, allergies (eczema, urticarial), bacterial, viral and parasitic infections, psoriasis, rosacea, stretch marks, vitiligo, hair loss and seborrhea, problems of pigmentation, lines and wrinkles, the improvement of skin texture, facial regeneration, the increasingly frequent use of invasive dermatological procedures such as peelings, laser treatments, etc.

The past few years have seen significant progress in the study of skin which has enabled the considerable development of skin evaluation and repair technologies.

Despite this remarkable progress, treatment options, for dermatological or cosmetic applications, are limited to the use of products developed and formulated for a specific application and commercialized by renowned companies in the field of dermatology and cosmetics.

Certain preparations may be prescribed by dermatologists and prepared extemporaneously by dispensing chemists.

However, the global supply of skin care and treatment today does not allow a personalized treatment to be formulated that adapts different active substances, different concentrations of such active substances and the synergy of successive treatments for a limited duration.

A strong tendency is developing in cosmetics and dermatology; that of bespoke skin care.

Patent EP-2343692 discloses an appliance for the formulation of bespoke cosmetics. However, this appliance may only be used for mixing and distributing purely cosmetic compositions such as nail polish, mascara, etc. A skin diagnosis is unnecessary and the appliance functions automatically. Moreover, the formulations prepared according to patent EP-2343692 require cumbersome apparatus. Lastly, there are no detailed examples of compositions.

Diagnosis prior to formulation, linked to new evaluation technologies, is one of the initiatives put in place to offer formulations that are adapted to the patient's skin and to the pathology from which the patient suffers. Cosmetic or dermatological formulations must thus be able to be performed strictly for the skin's specific need or needs.

Application WO-0191600 discloses a process for the manufacture of bespoke compositions that uses a prior diagnosis of the patient's skin, an appliance to mix the selected products and the supply of this mixture. However, the technical means to manufacture these cosmetic compositions are not explained, such as, for example, the chemical nature of the components.

Patent FR-2902995 discloses a cosmetic or dermatological composition in the form of an emulsion. The emulsion is manufactured extemporaneously by mixing a pulverulent phase composed of an oil and a porous solid with a hydrophilic, generally aqueous, phase. The problem with this composition lies in that it is only able to contain a limited number of active substances. Indeed, the active substance must be solubilised in the hydrophilic phase, thereby restricting its conditioning because of the risk of deterioration and bacterial growth, or be incorporated into the pulverulent phase, which may cause problems of stability, homogenization or formulation of this pulverulent phase. Moreover, the pulverulent phase and the hydrophilic phase must be adapted so as to manufacture the emulsion extemporaneously, thereby limiting the number of active substances that may be employed. Lastly, the cosmetic or dermatological composition according to patent FR-2902995 does not allow the quantity or nature of the active substances to be adjusted extemporaneously, in particular when these active substances are to be incorporated into the manufacturing process of the pulverulent phase.

A so-called fresh cosmetic composition has made its appearance, which consists in mixing the active substances at the last minute so as to preserve their use on the skin, and this thanks to improvements in conservation processes (dehydrated products), such as dual-compartment packaging.

A ready-made formula is not able to adequately satisfy the changing needs of the skin. Current compositions, which contain a previously determined unmodifiable mixture of active substances do not allow any flexibility of use.

To date, there is no bespoke cosmetic composition, prepared extemporaneously, that allows the active substances to be incorporated in a manner that is both qualitatively flexible (type of skin problem), quantitively flexible (degree of severity of skin problem) and stable (stability of the cosmetic composition, in particular an emulsion, and preservation of the active substance).

The present invention aims to overcome these difficulties by proposing an extemporaneously-prepared product comprising, on the one hand, a universal base, and on the other, one or more active substances selected further to an individualized diagnosis of the patient's skin.

The invention thus relates to an extemporaneous product for cosmetic and/or dermatological treatment comprising, at least:
one or several active substances acceptable in the field of cosmetic and/or dermatological skin care, in pulverulent or liquid form,
and a universal base comprising:
   at least one oil having a mass content of α-linoleic acid greater than 15% and a weight ratio of omega-6 to omega-3 of less than or equal to 5,
   water,
   glycerine,
   one or several emulsifiers,
   at least one alcohol other than glycerine, acceptable in cosmetic and/or dermatological compositions,
   at least one biomimetic peptide of the general formula A-X-Gly-His-Lys-Y in which:
      A represents a hydrogen atom or a radical of a monocarboxylic acid of the general formula R—COOH in which R represents an aliphatic radical comprising one to eleven carbon atoms,
      X represents a single bond, lysine, arginine or ornithine,
      and Y represents a group —OH or NH2,
         said universal base being able to solubilise said active substance or substances.

The percentage in mass of the active substance may vary from 0.25 to 30%. It goes without saying that the percentage of a specific substance depends on the effectiveness of the treatment defined. This percentage corresponds to a dosage whose effectiveness has been scientifically proven and, moreover, which is strictly defined in accordance with regulatory and toxicological constraints.

According to one characteristic of the invention, the oil present in the universal base has a mass concentration of between 5 and 25%, and preferably between 7.5 and 20%.

According to another characteristic of the invention, the oil is selected from among the following: camelina oil, also known as camelina seed oil, pumpkin seed oil, hemp oil, blackcurrant seed oil, flax seed oil, argan oil, olive oil, evening primrose oil, borage oil and perilla oil.

According to yet another characteristic of the invention, the glycerine present in the universal base has a mass concentration of between 1 and 20%, and preferably between 1 and 10%.

According to yet another characteristic of the invention, the universal base comprises at least one of the emulsifiers whose EU INCI name is as follows: sodium acrylate/sodium acryloyldimethyl taurate copolymer, polysorbate 80, xantham gum.

According to yet another characteristic of the invention, the universal base comprises one or several emulsifiers with a total mass concentration of between 1 and 5%.

According to yet another characteristic of the invention, the other alcohol present in the universal base has a mass concentration of between 10 and 30%, and preferably between 15 and 25%.

According to yet another characteristic of the invention, the other alcohol in the universal base is selected from the group formed by ethanol, propanol, propanediol, propan-2-ol, propylene glycol, methylene glycol or trimethylene glycol.

According to yet another characteristic of the invention, the biomimetic peptide present in the universal base has a concentration varying from 1 to 20 ppm, and preferentially from 3 to 10 ppm.

According to yet another characteristic of the invention, the universal base comprises at least one biomimetic peptide of the formula H-Gly-His-Lys-OH.

According to yet another characteristic of the invention, the universal base additionally comprises hyaluronic acid, preferentially at a mass concentration of less than 5%.

According to yet another characteristic of the invention, one or several active substances are selected from the group formed by ferulic acid, phloretin, SOD or superoxide dismutase, retinol and/or other retinoids, niacinamide or Vitamin B3, DMAE or dimethyl aminoethanol, resveratrol, isoflavones rich in genistein and daidzein, phytic acid, kojic acid, glycolic acid, arbutin, azelaic acid, N-acetyl cysteine, tyrosine, alpha-lipoic acid, vitamin C, vitamin B5, ceramides, hyaluronic acid, zinc and magnesium salts, salicylic acid, plant extracts rich in rutin, aloe vera, the madecassosides, caffeine, organic silicon, L-Carnitine, lactopeptides and/or bacteriocins, urea, lactic acid, mandelic acid, ursolic acid, resorcinol, hydroquinone and its derivatives, and/or other plant extracts rich in flavonoids that can have a beneficial effect on the skin.

According to yet another characteristic of the invention, one or several active substances are selected from among the families of the group formed by flavonoids, chalcones, zinc, calcium and magnesium salts of gluconic acid, vitamins and alpha and beta-hydroxy acids.

Another object of the invention relates to a kit for the extemporaneous preparation of a cosmetic and/or dermatological composition implementing an extemporaneous product, wherein it is constituted by a first sealed container containing the universal base, and a second sealed container containing at least one active substance, the two containers being able to be reversibly joined together and comprising means to combine their content by a simple manipulation or pressure.

A first advantage of the invention lies in the possibility of extemporaneously preparing a large number of cosmetic and/or dermatological formulations.

Another advantage of the invention lies in the stability over time of the universal base on the one hand and the active substances on the other.

Yet another advantage of the invention lies in the simplicity with which the cosmetic and/or dermatological compositions may be extemporaneously prepared.

Yet another advantage of the invention lies in the adjustment of the nature and quantity of each active substance respectively according to the type of skin condition or physiological need and the severity of such condition or need.

Yet another advantage of the invention lies in the possibility of obtaining extemporaneous preparations in a simple manner, which is to say bespoke preparations made by a pharmacist, an aesthetic doctor, plastic surgeon or dermatologist, possibly on the basis of a recommendation and/or prescription made by a doctor, for the treatment or skin care of a given patient, namely for a pathology (e.g. acne, stretch marks) and/or need (e.g. sagging skin, wrinkles and fine lines, cellulite), and for therapeutic properties (e.g. depigmentation).

Yet another advantage of the invention lies in the selection by the pharmacist, the doctor or the dermatologist of the skin treatment according to the actual skin disorder.

Yet another advantage of the invention lies in the wide range of skin conditions treated or skin needs fulfilled thanks to the presence, in the extemporaneously prepared skin compositions, of biomimetic peptides having a biological action on the one hand, and traditional active substances having a dynamic action on the other.

Other characteristics, particulars and advantages of the invention will be better understood from the additional description given by way of example with reference to the appended drawings, in which:

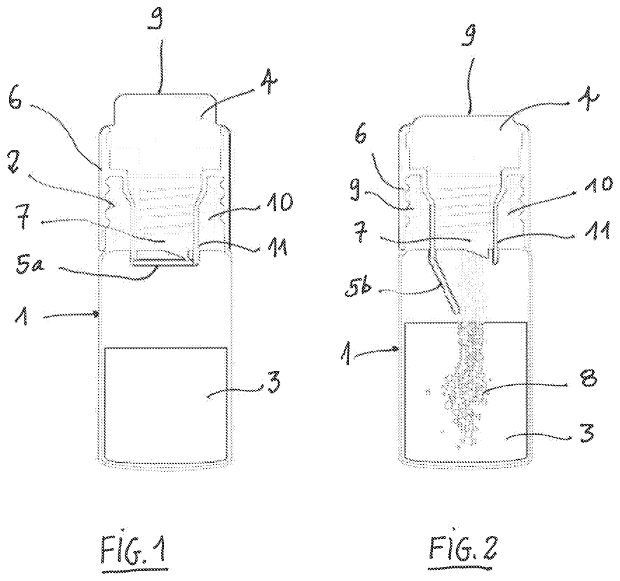

FIG. 1 shows a vial containing the universal base with a screw cap containing an active substance, and

FIG. 2 shows a vial containing the universal base with a cap releasing an active substance into the vial.

The invention is intended for the use of the extemporaneous product by dispensing chemists, dermatologists, general medical practitioners or aesthetic doctors for the preparation of cosmetic and/or dermatological compositions prepared extemporaneously according to the dermatological and/or cosmetic need of the patient.

The applicants have thus discovered, after long and patient research, an extemporaneous product comprising a universal base, on the one hand, and one or several active substances on the other. Said base comprises a specific choice of an alcohol, an oil and water so as to solubilise a great variety of solid or liquid active substances.

“Extemporaneous product” is understood to mean a product made of two parts, comprising firstly the universal base and secondly one or more pure and/or diluted active substances, this extemporaneous product being intended for the extemporaneous preparation of a cosmetic and/or dermatological composition.

“Universal base” is understood to mean a composition able to solubilise a great number of sold or liquid active substances and comprising at least one oil rich in polyunsaturated essential fatty acids and more particularly in omega-3, water, glycerine, at least one alcohol other than glycerine acceptable in the field of cosmetic and/or dermatological skin care, one or several emulsifying agents, and at least one biomimetic peptide. The universal base also has good stability over time, thereby facilitating its conservation for the extemporaneous preparation of a composition. The universal base may namely be an emulsion.

This universal base is able to solubilise the different, solid or liquid, active substances and still remain stable at least for 30 days, which represents twice as long as the prescribed period of use of the extemporaneously prepared composition, which is thus from 15 to 30 days.

The base has the considerable advantage of fulfilling all the practitioners' requirements in terms of the choice of treatment recommended in that he himself defines the active substance and dose to be incorporated. It is no longer necessary for the base to be adapted to the active substance. Contrary to the present state of the art, it is no longer necessary and restrictive to choose the support base according to the nature of the active substance. Indeed, cosmetic compositions available for sale always proceed by unique blends with no possibility for intervention or improvement.

Thus, a solid active substance, for example vitamin C, had to be incorporated into a base enabling its dissolution and in limited percentages. A hydrophobic or hydrophilic active substance had to be implemented with a compatible base.

The invention also confers a considerable advantage from an economic point of view since it is no longer necessary for the bases currently available on the market to be prepared, stored or modified so as to prepare a predetermined composition. Making a unique universal base available to the practitioner eliminates his need for a wide variety of bases whose conservation and period of use have to be monitored.

“Oil rich in polyunsaturated essential fatty acids” and more particularly omega-3 is understood to mean oil which has a high mass content of linolenic acid, that is to say at least 15%, and a relatively low weight ratio of omega-6 to omega-3, that is to say less than or equal to 5.

The oil described above not only allows certain active substances to be solubilised, but also has a recognized beneficial effect on the skin since it promotes the restoration of the hydrolipidic film and the production, in certain conditions, of cutaneous omega-3.

Within the scope of the invention, the oil present in the composition may namely be one of the following oils: camelina oil, also known as camelina seed oil, pumpkin seed oil, hemp oil, blackcurrant seed oil, flax seed oil, argan oil, olive oil, evening primrose oil, borage oil and perilla oil. All these oils possess the properties previously described.

Within the scope of the invention, the water present in the universal base is water habitually used in cosmetics or dermatology. It may be distilled water, demineralized water, or on the contrary, mineral water, etc.

Glycerine, or glycerol, of the formula HOH₂C—CHOH—CH₂OH, is a well-known polyol in the field of cosmetics, namely enabling failings in certain of the skin's lipid functions to be overcome. Amongst other things, glycerine enables better hydration, restoration of the natural defences, regulation of desquamation, improvement in the skin's elasticity and repair of the epidermis.

“Other alcohol” is understood to mean any alcohol, with the exception of glycerine, commonly used in cosmetics and/or dermatology, such as ethanol, propanol, propanediol, propan-2-ol, propylene glycol, methylene glycol or trimethylene glycol.

“Emulsifying agents” are understood to mean all and any compounds that favour emulsion, such as surfactants, anti-caking agents, stabilising agents, etc. These may namely be (EU INCI names): sodium acrylate/sodium acryloyldimethyl taurate copolymer, polysorbate 80, xantham gum. These three compounds may also be incorporated together into the universal base.

The universal base may be, for example, a gel or complex emulsion (triple emulsion, nanoemulsion, microencapsulation, microfluid, etc).

Within the scope of the invention, the biomimetic peptides used are of the general formula A-X-Gly-His-Lys-Y. A represents a hydrogen atom or a radical of a monocarboxylic acid of the general formula R—COOH in which R represents an aliphatic radical comprising one to eleven carbon atoms. X represents a single bond, lysine, arginine or ornithine. Y represents a group —OH or NH2. Lastly, Gly, His and Lys respectively represent glycine, histidine and lysine. Peptides are typically present in the universal base in proportions of 1 to 20 ppm, preferentially 3 to 10 ppm.

One of the preferred peptides corresponds to the formula: H-Gly-His-Lys-OH.

Several peptides, such as those sold by companies such as, for example, Lucas Meyer, Unipex, Mibelle, Caregen, Sederma may be combined in the universal base. The trade names of the peptides sold by these companies are, for example, Kollaren®, Thymulen®, Melitane®, ECM Protect®, B-White, etc. This allows a wide range of skin needs to be covered.

Biomimetic peptides are powerful active substances used in cosmetics and in dermatology. They specifically stimulate certain defective skin functions. Thus, the universal base is in itself cosmetically and/or dermatologically active. Thus, it is possible for different universal bases to be prepared each comprising one or several biomimetic peptide without the stability of the universal base being affected.

Optionally, the universal base may additionally comprise hyaluronic acid, for its lubricating and moisturising effect, at a mass concentration of preferably less than 5%.

The universal base itself has a cutaneous activity thanks in part to the biomimetic peptides. It may thus target various skin pathologies and disorders. A universal base may thus be prepared for all the skin pathologies, disorders or needs encountered by professionals in the field, and then their effects strengthened, potentiated or completed by means of different active substances, namely those mentioned above.

The universal base namely allows a very large number of active substances and families of active substances, for example solid substance such as vitamin C, to be solubilised.

Among the active substances that may be used, there are:
ferulic acid,
phloretin,
SOD or superoxide dismutase,
retinol and/or other retinoids,
niacinamide or Vitamin B3,
DMAE or dimethyl aminoethanol,
resveratrol,
isofalvones rich in genistein and daidzein,
phytic acid,
kojic acid,
glycolic acid,
arbutin,
azelaic acid,
N-acetyl cysteine,
tyrosine,
alpha-lipoic acid,
vitamin C,
vitamin B5,
zinc and magnesium salts,
salicylic acid,
plant extracts rich in rutin,
aloe vera,
madecassosides,
caffeine,
organic silicon,
L-Carnitine,
lactopeptides and/or bacteriocins,
urea,
lactic acid,
mandelic acid,
ursolic acid,
resorcinol,
ceramides,
hyaluronic acid,
hydroquinone and its derivatives,

and/or other plant extracts rich in flavonoids that can have a beneficial effect on the skin.

The active substances used in the invention have a high degree of purity and may be pulverulent or liquid.

Optionally, they may be dispersed in galenic products such as anti-caking excipients, thinners, etc.

The extemporaneous preparation of cosmetic and/or dermatological compositions may be performed in a simple manner by providing a kit, for example formed of a cap and a vial, both forms of packaging being sealed before use.

The cap contains one or several active substances. The vial contains the universal base.

The cap is able to be screwed onto the vial. When screwed onto the vial, simply pressing on the top of the cap is enough to break a membrane and release the active substance into the universal base. The solubilisation of the active substance in the universal base is performed by simply shaking the vial, optionally a specific appliance may be used.

FIG. 1 shows a vial 1 containing the universal base 3 and an upper part 10 provided with a threading 2 on its external edges, this vial 1 being screwed to a hollow cap 4 containing the active substance 8. The cap 4 has a tapping 6 that matches the threading 2 on the vial 1 and a membrane 5a in a closed position that ensures the sealing of the cap.

When the upper part 9 of the cap 4 is pressed, the active substance 8 is poured into the vial 8 ad is thus mixed with the universal base 3.

FIG. 2 shows the same vial 1 screwed with a cap 4 as in FIG. 1 but after pressure has been exerted on the upper part 9 of the cap 4. The membrane is thus broken by an element 7 in the lower part 11 of the cap, thereby allowing the active substance 8 to be released into the vial 1 and to mix with the universal base 3.

Several additional single doses of the same active substance may be added so as to increase the effect of the extemporaneously prepared cosmetic and/or dermatological composition, or several complementary active substances may be chosen, or else active substance with different effects may be chosen. Several tens of predetermined caps each containing one or several active substances may thus be made available by the pharmacist or the doctor.

In order for the quantities of the active substances introduced in the cap to be homogeneous, pure active substances may be dispersed within a pulverulent ingredient such as starch, dextran, aerosol, etc. This ingredient may be liquid. The active substances in the cap are in their purest form and are at an optimal concentration. The concentration varies according to the active substance in question and complies with applicable legislation.

The aid of a health professional such as a pharmacist, a cosmetic doctor or a dermatologist may prove necessary. This aid is indeed recommended for the precise diagnosis of the skin, and then the choice and dosage of one or several active substances, the homogenisation of the mixture and the recommendations for use to the consumer (e.g. recommended dosage). Optionally, the health professional may perform the skin diagnosis with the aid of one of the following appliances: Cornéomètre®, Cutomètre®, Mobile Skin Scope MSS Quantirides®, Sébumètre®, Mexamètre®, Dermalab® etc.

All the concentrations expressed as a percentage in the present application are expressed as a mass percentage.

Within the general scope of the invention, the following skin disorders may namely be treated: acne, rosacea, seborrheic oily skin, seborrheic dermatitis, sensitive skin and/or skin made sensitive after mechanical acts like dermabrasion, liquid nitrogen (N2) and laser burns and/or further to invasive acts like hyaluronic acid injections/mesotherapy, redness, itching, psoriasis, wound healing, accelerated and/or chronological aging, peeling, skin inflammation, effects of UV radiation, blackheads, excess sebum, enlarged pores, age spots, hyperpigmentation, irritation, couperose, atopic dermatitis, bacterial or viral infections, loss of firmness, lines and wrinkles, stretch marks, cellulite, etc. dermatoporosis, etc.

The application on the skin of cosmetic and/or dermatological preparations will preferably be performed with a dropper and/or by hand.

A certain number of cosmetic and/or dermatological compositions have been prepared extemporaneously in accordance with the following formulations. The concentrations are always by mass. The percentages of commercial peptides in the universal base do not correspond to the percentage of pure peptides, since “commercial peptides” is understood to mean formulations in which the different peptides are commercialised, the final concentration of the pure peptide always being of between 1 and 20 ppm.

### EXAMPLE 1

To illustrate one example embodiment of the invention, a cosmetic composition is produced containing the biomimetic peptide Kollaren® in order to treat a depigmentation effect, to which is added a cap containing 390 mg that is 3%, of the active substance arbutin.

| | % in mass |
|---|---|
| Universal base | |
| Camelina oil | 12 |
| Ethanol | 24 |
| Glycerine | 2.5 |
| Emulsifier | 3 |
| Commercial peptide: | 2 ppm |
| Kollaren ® | |
| Water | qs |
| Active Substance | |
| Arbutin | 3 |

### EXAMPLE 2

To treat a patient suffering from a slight pigmentation disorder, two active substances are added to a vial containing 13 ml of the composition according to Example 1 by means of a cap containing 520 mg of Vitamin B3 (that is 4% of Vitamin B3 for the whole composition) and 3% in mass of arbutin.

| | % in mass |
|---|---|
| Universal base | |
| Camelina oil | 12 |
| Ethanol | 24 |
| Glycerine | 2.5 |
| Emulsifier | 3 |
| Commercial peptide: | 3 ppm |
| Kollaren ® | |
| Water | qs |
| Active Substance | |
| Arbutin | 3 |
| Vitamin B3 | 4 |

### EXAMPLE 3

To treat a patient suffering from a serious hyperpigmentation skin disorder, an additional active substance is added by means of a cap 3% of arbutin to the vial containing the composition according to Example 2. Arbutin is thus added twice to prepare the composition extemporaneously.

The following composition is thus obtained:

| | % in mass |
|---|---|
| Universal base | |
| Camelina oil | 12 |
| Ethanol | 24 |
| Glycerine | 2.5 |
| Emulsifier | 3 |
| Commercial peptide: | 6 ppm |
| Kollaren ® | |
| Water | qs |
| Active Substance | |
| Arbutin | 6 |
| Vitamin B3 | 4 |

### EXAMPLE 4

| | % in mass |
|---|---|
| Universal base | |
| Camelina oil | 10 |
| Ethanol | 20 |
| Glycerine | 2 |
| Emulsifier | 1.75 |
| Commercial peptides | 11 ppm |
| Kollaren ® GL 5000 ppm | |
| Melitane ® GL 1000 ppm | |
| Thymulen ® 47 GL 1000 ppm | |
| ECM Protect ® GL 1000 ppm | |
| Water | qs |
| Active Substance | |
| Vitamin C | 14 |
| SOD | 2 |

### EXAMPLE 5

| | % in mass |
|---|---|
| Universal base | |
| Camelina oil | 8 |
| Ethanol | 12 |
| Glycerine | 1.2 |
| Emulsifier | 4 |
| Kollaren ® peptides | 15 ppm |
| GL 5000 ppm | |
| Water | qs |
| Active Substance | |
| Kojic acid | 2 |

### EXAMPLE 6

| | % in mass |
|---|---|
| Universal base | |
| Camelina oil | 6 |
| Ethanol | 16 |
| Glycerine | 2 |
| Emulsifier | 2 |
| Melitane ® GL 1000 | 18 ppm |
| ppm peptides | |
| Hyaluronic acid | 0.3 |
| Water | qs |
| Active Substance | |
| Vitamin B5 | 5 |

### EXAMPLE 7

| | % in mass |
|---|---|
| Universal base | |
| Camelina oil | 15 |
| Ethanol | 10 |
| Glycerine | 4 |
| Emulsifier | 4.8 |
| Melitane ® GL 1000 | 2 ppm |
| ppm peptides | |
| Water | qs |
| Active Substance | |
| Arbutin | 3 |

### EXAMPLE 8

| | % in mass |
|---|---|
| Universal base | |
| Camelina oil | 23 |
| Ethanol | 28 |
| Glycerine | 9 |
| Emulsifier | 2 |
| Melitane ® GL 1000 | 5 ppm |
| ppm peptides | |
| Water | qs |
| Active Substance | |
| Arbutin | 6 |

### EXAMPLE 9

| | % in mass |
|---|---|
| Universal base | |
| Camelina oil | 10 |
| Ethanol | 20 |
| Glycerine | 15 |
| Emulsifier | 2 |
| Melitane ® GL 1000 | 7 ppm |
| ppm peptides | |
| Water | qs |
| Active Substance | |
| Arbutin | 9 |

### EXAMPLE 10

| | % in mass |
|---|---|
| Universal base | |
| Camelina oil | 10 |
| Ethanol | 20 |
| Glycerine | 15 |
| Emulsifier | 2 |
| Melitane ® GL 1000 | 2 ppm |
| ppm peptides | |
| Water | qs |
| Active Substance | |
| Arbutin | 3 |
| Aloe Vera | 5.4 |
| Vitamin C | 7 |

### EXAMPLE 11

| | % in mass |
|---|---|
| Universal base | |
| Camelina oil | 10 |
| Ethanol | 20 |
| Glycerine | 15 |
| Emulsifier | 2 |
| Melitane ® GL 1000 | 2 ppm |
| ppm peptides | |
| Water | qs |
| Active Substance | |
| Arbutin | 3 |
| Vitamin C | 7 |

### EXAMPLE 12

| | % in mass |
|---|---|
| Universal base | |
| Camelina oil | 10 |
| Ethanol | 20 |
| Glycerine | 15 |
| Emulsifier | 2 |
| Melitane ® GL 1000 | 3 ppm |
| ppm peptides | |
| Water | qs |
| Active Substance | |
| Vitamin C | 7 |

### EXAMPLE 13

| | % in mass |
|---|---|
| Universal base | |
| Camelina oil | 12 |
| Ethanol | 24 |
| Glycerine | 2.5 |
| Emulsifier | 3 |
| Kollaren ® commercial | 3 ppm |
| peptide | |
| Water | qs |
| Active Substance | |
| Arbutin | 6 |
| Vitamin B3 | 4 |
| Vitamin C | 7 |

### EXAMPLE 14

| | % in mass |
|---|---|
| Universal base | |
| Camelina oil | 12 |
| Ethanol | 24 |
| Glycerine | 2.5 |
| Emulsifier | 3 |
| Kollaren ® commercial | 3 ppm |
| peptide | |
| Water | qs |
| Active Substance | |
| Vitamin B3 | 4 |
| Zinc sulphate | 5 |

The main constituents of the universal base are indicated above, but it goes without saying that this list is completed by the addition of the usual ingredients used in the field of cosmetics at low percentages. These ingredients are intended to confer certain properties to the creams and gels prepared hereafter, such as for example, texture. By way of example, 0.6% of SYMDIOL® 68, 1.5% of SIMULGEL®, or else 0.25% of RHODICARES S® may be used.

In the examples above, arbutin, Vitamin B3, Vitamin C, SOD, kojic acid, aloe vera and Vitamin B5 have been used, but it goes without saying that all the active substances previously indicated may be used in the proportions indicated with the same bases. The choice of a single or compound substance depends on the skin condition to be treated and the severity of this condition. The application of a day preparation may be combined with the application of a night preparation, or preparations for the eye contour, the hands and décolleté with different proportions of the active substance. The prescribing practitioner remains free to define the composition to be implemented according to the diagnosis he has established.

It is known for arbutin to inhibit the production of melanin, the pigment responsible for the colouration of the skin. It lightens the skin, reduces the depth of any patches or blotches and evens out the skin tone. Trials performed with a composition according to Example 1 show that after 30 days of treatment, there is a clear lightening of such marks and after 60 days of treatment such marks have almost disappeared.

Thus, for a skin condition linked to UV (helidermititis), a preparation may be produced that is constituted by 14% of Vitamin C and 2% of SOD (Example 4) for application during the day and a preparation constituted by 6% of arbutin, 4% of Vitamin B3 and 7% of Vitamin C (Example 13) for day and night application. It is also known that Vitamin C protects the skin from damage caused by free radicals due to environmental stress (pollution, UV), stimulates the synthesis of collagen and the renewal of fibroblasts, inhibits tyrosinase and evens the skin tone.

There again, trials performed with the composition according to Example 13 show that after 60 days the skin is clear and remodelled.

Lastly, the trials performed with the composition according to Example 14 show that after 7 days of treatment the skin is lightened and there is a reduction in acne pimples.

The same is true of aloe vera which helps to soothe fragile skin and reduces inflammation. It has an enzymatic action which is slightly exfoliating and astringent.

The known properties of these three active substances mentioned above make it easier to understand the interest of the invention wherein these active substances may be combined using a single base.

The trials performed using compositions according to Examples 1 to 14 have given excellent results in the treatment of various skin conditions or in response to different skin needs. These compositions have excellent stability and storage at room temperature for 30 days leads to no deterioration of these compositions. Moreover, no loss of effectiveness has been observed in compositions thus prepared.

The compositions in Examples 1 to 14 have each been prepared in the same way but replacing the camelina oil by the following oils: pumpkin seed oil, hemp oil, flax seed oil and perilla oil.

In the same way, each of the compositions according to Examples 1 to 14 has been prepared but replacing the ethanol by one of the following alcohols: propanol, propanediol, propan-2-ol, propylene glycol, methylene glycol or trimethylene glycol. No noticeable modification in the compositions prepared has been observed.

Stability, effectiveness and deterioration tests performed on the modified compositions have produced the same results as those obtained for compositions using camelina oil. The same is true when the ethanol is replaced by the other alcohols mentioned above.

Thus, for the first time, the invention allows any cosmetic and/or dermatological composition to be produced from active substances by enabling their dissolution and solubilisation in oil, or water, or alcohol. Thus, the practitioner has a single base from which he prepares a specific composition for the diagnosed condition to be treated which uses either a single active substance freely dosed according to the severity of the condition, or a combination of several active substances.

This composition is all the easier to prepare in that the practitioner has a range of pre-dosed active substances at his disposal which require little adaptation.

As mentioned previously, the universal base implemented in the invention in itself produces a beneficial effect on the skin, contrary to all the commercially-available bases which generally have no effect.

The skin healing effect of this preparation has thus been analysed. A model to alter the skin's reconstructive capacities was made by applying a dermocorticoid to the surface of the epidermis so as to quantify the stimulation of cellular renewal. This experimental model is pertinent, since it approaches the secondary effects observed by dermatologists after the repeated application on the skin of dermocorticoids whose consequence is a thinning of the skin, increased fragility and sensitivity to infections. The stimulation of cellular renewal was studied by the quantification of the mitotic potential of the basal cells.

The moisturising and restructuring effect of the preparation has been analysed after the production of a model to alter the skin barrier using sodium lauryl sulphate (SLS) responsible for transepidermal water loss and expression of involucrin (protein acting as a structural foundation for the protein of the stratum corneum and detected in the cytoplasm of the stratum granulosum and the upper part of the squamous cell layer). Evaluating the involucrin expression allows the protection provided by the cream to be quantified with respect to the stratum corneum and the stimulation of its metabolism with respect to the skin's keratinocytes.

The soothing effect of the preparation has been analysed after the production on the skin of an experimental burn model (application of an agarose gel at 80° C.). Its soothing activity was studied by analysing the limitation both of the destruction of the epidermis (corresponding to the extension f the burn) and the vascular dilation (method of analysing vessels: Branchet et coll, Skin Pharmacol Appl Skin Physiol 1999; 12/211-220).

To this end, a base has been prepared containing 64% of water, 20% of alcohol, 10% of camelina oil, 2% of glycerine, 1.5% of SIMUGEL® EG, 0.6% of SYMDIOL® 68, 0.001 ppm of Kollaren® and various additives used in the cosmetic field at percentages of less than 2%, such as for example, emulsifiers of the type: sodium acrylate/sodium acryloyldimethyl taurate copolymer, polysorbate 80, xantham gum.

### EQUIPMENT AND METHODS

1) Assessment of the Healing Effect of the Preparation

a) Organ Cultures, Maintenance in Survival Conditions

Skin fragments from 8 different donors were cultured for 4 days with an application of class II dermocorticoid (Diprosone® cream containing betamethasone at 0.05%) on DO and D1 allowing a reduction in the mitotic index to be obtained.

The preparation was applied every day from D1 to D4.

b) Immunohistochemical Demonstration of Mitotic Cells (Ki67).

The healing effect analysis was assessed by quantifying the mitotic cells. The skin fragments were fixed in Bouin's fluid and embedded in paraffin. The epithelial proliferation was analysed by immunohistochemistry using an anti-Ki67 antibody (marker for cells in M, S, G1 and G2 phases of the cell cycle). Immunodetection was performed using a 3-layer indirect immunoperoxidase technique, amplified (kit CsA, DAKO) and revealed by AEC (3-amino-9-ethylcarbazole).

A stained cell count was performed and divided by the total count of basal cells to calculate the percentage of stained cells.

Results
Control skin: 12±6.2%
Skin altered by dermocorticoid: 7.5±2.5%
Skin altered and treated: 11.7±3.2%

A significant improvement can be observed in the altered skin. In the experimental model where the skin is altered by dermocorticoids, a decrease in the mitotic index is demonstrated close to significance (p=0.075) with a percentage of 7.5 compared with 12% for the control skin samples.

After treatment using the preparation, a significant recovery of this proliferation index is observed with a percentage of stained cells of 11.7% in comparison with the experimentally altered skins (p=0.017).

2) Assessment of the Moisturising and Restructuring Effect of the Preparation

a) Organ Cultures, Maintenance in Survival Conditions

Fragments of skin from 8 different donors were kept alive for 4 days with 2 topical applications of SLS at 1% (sodium lauryl sulphate) at DO and D1 allowing an alteration in the skin barrier to be obtained. The Gel Cream was applied every day from D1 to D4.

b) Immunohistochemical Demonstration of Involucrin

The skin fragments were fixed in Bouin's fluid and embedded in paraffin. Immunodetection was performed using a 3-layer indirect immunoperoxidase technique (ABC Peroxidase kit, Vector Laboratories) and revealed by AEC.

The intensity of Immunohistochemical staining of the stratum granulosum and the squamous cell layer was assessed by the expression of staining using a semi-quantitative histological score:

RESULTS (Intensity Score)
Control skin: 2.4±0.8%
Skin altered by SLS: 1.4±1.2%
Skin altered and treated: 3±0.9%.

A statistically significant difference can be observed with respect to the control skin samples, with P<0.05.

3) Assessment of the Soothing Effect of the Preparation

Score 0: negative staining

Score 1: low staining intensity

Score 2: moderate staining intensity

Score 3: high staining intensity

Score 4: very high staining intensity.

The staining topography with respect to the epithelium was also analysed by verifying any staining in the stratum granulosum and the squamous cell layer using a semi-quantitative histological score:

Score 1: granular layer of the epithelium

Score 2: upper third of the epithelium

Score 3: two thirds of the epithelium

Score 4: three quarters of the epithelium (all the epithelium except for the basal layer).

Results (topographical score)
Control skin: 2±0.8%
Skin altered by SLS: 1.7±1.5
Skin altered and treated: 2.5±1

A significant decrease (p=0.04) in the expression of involucrin staining is demonstrated after alteration by SLS: intensity score of 1.4 with respect to the control skin sample (score of 2.4).

After treatment using the preparation, a significant increase is observed in involucrin expression: score of 3 as opposed to 1.4 with respect to the altered skin (p=0.003).

A decrease (not significant) is demonstrated after SLS of the distribution of the involucrin within the epithelium with a score of 1.73 as opposed to 2 for the control skin. An increase (also not significant) of its distribution is observed after treatment using the preparation with a score of 2.5.

4) Assessment of the Soothing Effect of the Preparation

a) Organ Cultures, Maintenance in Survival Conditions

Skin fragments from 8 different donors for four days. The experimental model of epithelial alteration was obtained by burning after application of an agarose gel at 85° C. on DO. Immediately afterwards, the Gel Cream was applied every day up to D4.

The soothing effect was assessed both by analysis of the limitation of the alterations of the epithelium and of the vasodilation.

b) Histological Analysis of the Epithelium

The skin fragments were fixed in Bouin's fluid and embedded in paraffin.

After colouration with hemaluneosin, the level of alteration of the epidermis was assessed: eosinophilic clearing or necrosis of the cytoplasm, cleared or pyknotic nuclei. Thus, the number of cells having signs of extensive necrosis or slight clearing of the cytoplasm or the nucleus was noted, for each field analysed. For each skin sample, the percentage of altered cells was calculated (10 fields with a magnification of 40 on three different sections) by deducting the percentage of cells altered by the maintenance in survival conditions (albeit slight) observed on the control skin samples.

c) Analysis of the Vasodilation and Oedema

The skin fragments were fixed in Bouin's fluid and embedded in paraffin. After colouration with hemaluneosin, the soothing effect was measured by morphometric quantification of the surface occupied by capillaries, the percentage of dilated capillaries and by the amount of oedema in the upper dermis.

After colouration with hemaluneosin, the vascular dilation is assessed by counting the number of dilated vessels over the whole of the histological section (16 fields at a magnification of 40). This number is divided by the total number of vessels so as to calculate the percentage of dilated vessels. Furthermore, a morphometric analysis of the surface (μm²) occupied by the vessel lumen is performed to determine the mean surface (μm²) occupied in the dermis by the vessels.

The assessment of the oedema is performed using semi-quantitative scores:
Absence of oedema: 0 (score 0)
Slight oedema: + (score 1)
Moderate oedema: ++ (score 2)
Severe oedema: +++ (score 3)

A comparative study was performed between:
control skin
skin altered by burning
skin altered and treated by the Gel Cream

d) Expression of the Results and Statistical Analysis

A comparative study of the results between the skins treated using the cream and those not treated (control skins) or test skins (alteration model by dermocorticoid, SLS or burning) was performed from the means scores ±SD.

The statistical analysis was performed using the paired Student's test with an alpha risk of 5%.

Results

Skin+burn: 24.1±25.1%

Skin+burn+preparation: 15.8±11.6%

An increase in the alterations of the epidermis is observed after burning with a percentage of altered cells of 24.1%. A decrease in these not significant alterations after treatment using the cream is demonstrated with a percentage of altered cells of 15.8%.

5) Analysis of the Vasodilation

After burning, a dilation of the capillaries of the dermis was demonstrated with an increased percentage of dilated vessels of 99.3 as opposed to 87.6 for the control skin (p=0.004) and a mean surface of 270.8 μm² as opposed to 171.6 μm² for the control skin (p=4.6 0.10⁻⁵).

Treatment using the preparation enabled a significant reduction in the dilation of the capillaries with a mean surface of 197.8 μm² as opposed to 270.8 μm² for the test skin (p=0.003). The percentage of dilated vessels also significantly decreased to 91.25% p=0.02).

6) Analysis of the Oedema

A significant increase in the dermal oedema has been observed after burning with a score of 2.26 as opposed to 1.73 for the control skin (p=0.036).

A not significant decrease in the oedema has been demonstrated after treatment using the preparation with a histological score of 1.88.

### General Conclusions

The healing effect of the preparation was demonstrated by obtaining a statistically significant restoration of the mitotic index of the epithelium after experimental alteration of the skin.

The effects of the compositions comprising the active substance or substances were assessed in the same way.

The moisturising and restructuring effect of the preparation was visualised and quantified after the performance of a model to alter the skin barrier using sodium lauryl sulphate by demonstrating an increase in involucrin expression in the epidermis. The increase of this expression attests the protection provided by the cream but also the stimulation of the involucrin metabolism in the cells of the upper part of the epithelium.

The soothing effect of the preparation was significantly quantified after performance on the skin of an experimental burn model by demonstrating the limitation in dilation of the skin's capillaries. The limitation in the destruction of the epidermis (corresponding to an extension of the burn) was more moderately observed.

The incorporation of the active substance (single or multiple) in the base described and used as explained above allows the effect of this active substance to be sublimated and intensified. Naturally, the action of the composition comprising the universal base+active substance enables a faster and more beneficial action with respect to a composition comprising a conventional base with this same active substance.

1-14. (canceled)

15. A method for treating a subject with one or several active substances acceptable in the field of cosmetic and/or dermatological skin care in need thereof comprising:
providing a plurality of kits, each kit containing a universal base component and a different active substance component,
determining the need of a subject for treatment with an active substance component present in one of said kits,
selecting the one of said kits,
mixing the universal base component and the active substance component of the one of said kits to form a mixture, and
treating the subject with said mixture,
wherein the universal base component comprises:
at least one oil having a mass content of α-linoleic acid greater than 15% and a weight ratio of omega-6 to omega-3 of less than or equal to 5,
water,
glycerine,
one or several emulsifiers,
at least one alcohol other than glycerine, acceptable in cosmetic and/or dermatological compositions,
at least one biomimetic peptide of the general formula A-X-Gly-His-Lys-Y in which:
A represents a hydrogen atom or a radical of a monocarboxylic acid of the general formula R—COOH in which R represents an aliphatic radical comprising one to eleven carbon atoms,
X represents a single bond, lysine, arginine or ornithine,
and Y represents a group —OH or NH2,
said universal base being able to solubilise said active substance or substances and remain stable after solubilization for at least 30 days at room temperature, wherein
the oil has a mass concentration of between 5 and 25%,
the glycerine has a mass concentration of between 1 and 20%,
the one or several emulsifiers has a mass concentration of between 1 and 5%,
the at least one alcohol other than glycerine has a mass concentration of between 10 and 30%, and
the biomimetic peptide has a mass concentration varying from 1 to 20 ppm,
each of the above mass concentrations being based on the mass of the universal base, and
wherein the active substance component is acceptable in the field of cosmetic and/or dermatological skin care, in pulverulent or liquid form.

16. The method according to claim 15, wherein the oil present in the universal base has a mass concentration of between 7.5 and 20%.

17. The method according to claim 15, wherein the oil is selected from among the following: camelina oil, pumpkin seed oil, hemp oil, flax seed oil, and perilla oil.

18. The method according to claim 15, wherein the glycerine present in the universal base has a mass concentration of between 1 and 10%.

19. The method according to claim 15, wherein the universal base comprises at least one of the emulsifiers whose EU INCI name is as follows: sodium acrylate/sodium acryloyldimethyl taurate copolymer, polysorbate 80, xantham gum.

20. The method according to claim 15, wherein the other alcohol present in the universal base has a mass concentration of between 15 and 25%.

21. The method according to claim 15, wherein the other alcohol in the universal base is selected from the group formed by ethanol, propanol, propanediol, propan-2-ol, propylene glycol, methylene glycol or trimethylene glycol.

22. The method according to claim 15, wherein the biomimetic peptide present in the universal base has a concentration varying from 3 to 10 ppm.

23. The method according to claim 15, wherein the universal base comprises at least one biomimetic peptide of the formula H-Gly-His-Lys-OH.

24. The method according to claim 15, wherein the universal base additionally comprises hyaluronic acid.

25. The method according to claim 15, wherein one or several active substances are selected from the group formed by ferulic acid, phloretin, SOD or superoxide dismutase, retinol and/or other retinoids, niacinamide or Vitamin B3, DMAE or dimethyl aminoethanol, resveratrol, isoflavones containing genistein and daidzein, phytic acid, kojic acid, glycolic acid, arbutin, azelaic acid, N-acetyl cysteine, tyrosine, alpha-lipoic acid, vitamin C, vitamin B5, zinc and magnesium salts, salicylic acid, plant extracts containing rutin, aloe vera, the madecassosides, caffeine, organic silicon, L-Carnitine, lactopeptides and/or bacteriocins, urea, lactic acid, mandelic acid, ursolic acid, resorcinol, ceramides, hyaluronic acid, hydroquinone and its derivatives, and/or other plant extracts containing flavonoids.

26. The method according to claim 15, wherein one or several active substances are selected from among the families of the group formed by flavonoids, chalcones, zinc, calcium and magnesium salts of gluconic acid, vitamins and alpha and beta-hydroxy acids.

27. The method according to claim 15, wherein at least some of the kits differ from each other by containing a different active substance component.

28. The method according to claim 15, wherein each kit is constituted by a first sealed container containing the universal base component, and a second sealed container containing the active substance component, the two containers being able to be reversibly joined together and comprising means to combine their content by a manipulation or pressure.
